(19) United States
(12) Patent Application Publication
(10) Pub. No.: US 2024/0216076 A1
(43) Pub. Date: Jul. 4, 2024

(54) CATHETER ASSEMBLY TRACKING AND VISUALIZATION

(71) Applicant: Boston Scientific Scimed Inc., Maple Grove, MN (US)

(72) Inventors: Sarah Renee Gutbrod, St. Paul, MN (US); Thomas Ferree, Waltham, MA (US); Matthew Ferrante, Cambridge, MA (US); Jackie Leung, Richmond Hill, CA (US); Prateek Mathur, Toronto (CA)

(21) Appl. No.: 18/512,811

(22) Filed: Nov. 17, 2023

Related U.S. Application Data
(60) Provisional application No. 63/436,358, filed on Dec. 30, 2022.

(51) Int. Cl. A61B 34/20; A61B 18/14

(52) U.S. Cl. CPC A61B 34/20; A61B 18/1492; A61B 2018/00577

(57) ABSTRACT

A system for use with an electrophysiological procedure is disclosed. The system includes an elongated catheter assembly having a tracking sensor and a controller. The controller is configured to track catheter assembly positions within the organ based on a plurality of electrical signals from the tracking sensor, determine a first catheter assembly position based on a first electrical and determine a second catheter assembly position based on a second electrical signal, constrain the second position with the first position, and generate an anatomical map of an organ of the electrophysiological procedure with a visualization of the catheter assembly having the second position constrained with the first position.

### CROSS-REFERENCE TO RELATED APPLICATIONS

This application claims the benefit of U.S. Provisional Application No. 63/436,358, entitled “CATHETER ASSEMBLY TRACKING,” and filed Dec. 30, 2022, which is hereby incorporated by reference in its entirety.

### TECHNICAL FIELD

The present disclosure relates generally to medical systems and methods for electrophysiological procedures, such as ablating tissue, in a patient via catheter assemblies. More specifically, the present disclosure relates to medical systems and methods for tracking catheter assemblies within the patient during the electrophysiological procedure.

### BACKGROUND

Electrophysiological procedures involve guiding catheter assemblies into the heart and tracking the location of the catheter assemblies with respect to the heart. Catheter ablation is a minimally invasive electrophysiological procedure to treat a variety of heart conditions such as supraventricular and ventricular arrhythmia. Such procedures can involve the visualization of the heart, heart activity, and the position of the catheter assembly within the heart. A common visualization system involves the use of fluoroscopy, which can expose the patient and clinician to ionizing radiation. Electroanatomical mapping is an alternative visualization technique that does not involve the use of ionizing radiation. Electroanatomical mapping allows a clinician to accurately determine the location of an arrhythmia, define cardiac geometry in three dimensions, delineate areas of anatomic interest, and permits spatial localization of the catheter assembly for positioning and manipulation.

Catheter assemblies include a plurality of catheter elements such as catheters, sheaths, dilators, guidewires, and needles. For instance, a catheter assembly can include a catheter and a sheath. Navigation-enabled catheter assembly elements, such as navigation-enabled catheters, use magnetic fields to track a magnetic sensor within the catheter element with relative accuracy in electroanatomical mapping systems. But not all catheter elements include magnetic sensors. Impedance-based catheter elements, such as catheter sheaths or catheters, use electric fields to track the catheter element with lower cost components such as electrodes but are generally less accurate than navigation-enabled catheter elements in electroanatomical mapping systems. Regardless of whether the catheter element is navigation-enabled or impedance-based, electroanatomical mapping systems use electrical measurements to determine a catheter pose, or the three-dimensional curve of the distal portion of the catheter assembly spanned by electrodes.

### SUMMARY

In Example 1, a system for use with an electrophysiological procedure, the system comprising an elongated catheter assembly having a tracking sensor, and a controller. The controller is configured to track a plurality of catheter assembly positions within the organ based on a plurality of electrical signals from the tracking sensor, determine a first catheter assembly position based on a first electrical signal of the plurality of electrical signals and determine a second catheter assembly position based on a second electrical signal of the plurality of electrical signals, constrain the second position with the first position, and generate an anatomical map of an organ of the electrophysiological procedure with a visualization of the catheter assembly having the second position constrained with the first position.

In Example 2, the system of Example 1, wherein the controller is configured to place a tag on the anatomical map of the organ at the first catheter assembly position representing an anatomical landmark.

In Example 3, the system of Example 2, wherein the anatomical landmark is an inter-atrial septum.

In Example 4, the system of any of Examples 1-3, wherein the controller is configured to track the catheter assembly independent of the anatomical landmark.

In Example 5, the system of any of Examples 1-4, wherein the controller is configured to track the plurality of catheter assembly positions of the catheter assembly via the tracking sensor using a tracking methodology as a function of time as it passes through the heart.

In Example 6, the system of any of Examples 1-5, wherein the controller is configured to track the plurality of catheter assembly positions of the catheter assembly generally concurrently via the tracking sensor using a tracking methodology.

In Example 7, the system of any of Examples 1-6, wherein the first catheter assembly position includes a first location in space and a first tangent, and the second catheter assembly position includes a second location in space and a second tangent.

In Example 8, the system of any of Examples 1-7, wherein the controller is configured to constrain the first position to the second position via bending energy data regarding bending energy of the catheter assembly.

In Example 9, the system of any of Examples 1-8, wherein the controller is configured to constrain the first position to the second position via a path.

In Example 10, the system of Example 1, wherein the catheter assembly includes a plurality of coaxially disposed catheter elements, the catheter elements including a first catheter element and a second catheter element, the first catheter element forming an elongated lumen and the second catheter element disposed within the lumen, the first and second catheter elements movable with respect to each other along an axis, wherein the first catheter element includes a first tracking sensor and the second catheter element includes a second tracking sensor. The controller is configured to generate a first position of the first catheter element based on a first electrical signal of the plurality of electrical signals and generate a second position of the second catheter element based on a second electrical signal of the plurality of electrical signals, laterally align the first position with the second position based on a lateral displacement and a rotational displacement determined from the first position and the second position, and longitudinally adjust the first position with respect to the second position based on a detection of the first tracking sensor of the second tracking sensor.

In Example 11, the system of Example 10, wherein the first catheter assembly position includes a first location in space and a first tangent, and the second catheter assembly position includes a second location in space and a second tangent.

In Example 12, the system of Example 11, wherein the first location and the first tangent are laterally aligned with the second location and the second tangent based on a lateral departure and a rotational deflection determined from the first tangent and the second tangent.

In Example 13, the system of any of Examples 11-12, wherein the controller is configured to longitudinally adjust the first location with respect to the second location based on a sheath detection.

In Example 14, the system of any of Examples 10-13, wherein the controller is configured to track the first catheter element via impedance tracking and to track the second catheter element via magnetic tracking.

In Example 15, the system of any of Examples 10-14, wherein the controller is configured to laterally align the second position to coincide with the first position.

In Example 16, a system for use with an electrophysiological procedure, the system comprising an elongated catheter assembly and a controller. The elongated catheter assembly including a plurality of coaxially disposed catheter elements, the catheter elements including a first catheter element and a second catheter element, the first catheter element forming an elongated lumen and the second catheter element disposed within the lumen, the first and second catheter elements movable with respect to each other along an axis, wherein the first catheter element includes a first tracking sensor and the second catheter element includes a second tracking sensor. The controller is configured to determine an anatomical map of a heart of the electrophysiological procedure and track a catheter assembly position within the organ based on a first electrical signal from the first tracking sensor and based on a second electrical signal from the second tracking sensor, generate a first position of the first catheter element based on the first electrical signal and generate a second position of the second catheter element based on the second electrical signal, laterally align the first position with the second position based on a lateral displacement and a rotational displacement determined from the first position and the second position, and longitudinally adjust the first location with respect to the second location based on a detection of the first tracking sensor of the second tracking sensor.

In Example 17, the system of Example 16, and further including the controller configured to generate a visualization with the catheter elements with respect to the anatomical map of the heart after laterally aligning and longitudinally adjusting.

In Example 18, the system of Example 16, wherein the first catheter assembly position includes a first location in space and a first tangent, and the second catheter assembly position includes a second location in space and a second tangent.

In Example 19 the system of Example 18, wherein the first location and the first tangent are laterally aligned with the second location and the second tangent based on a lateral departure and a rotational deflection determined from the first tangent and the second tangent.

In Example 20, the system of Example 16, wherein the controller is configured to longitudinally adjust the first location with respect to the second location based on a sheath detection.

In Example 21, the system of Example 16, wherein the controller is configured to apply a confidence value to the second position.

In Example 22, the system of Example 21, wherein the confidence value is 1.0 if the second catheter element is magnetically tracked in an electrophysiology system.

In Example 23, the system of Example 16, wherein the first catheter element is a sheath and the second catheter element is a catheter disposed within the sheath.

In Example 24, a process for use with an elongate catheter assembly during an electrophysiological procedure on the heart. The catheter assembly including a plurality of coaxially disposed catheter elements, the catheter elements including a first catheter element and a second catheter element, the first catheter element forming an elongated lumen and the second catheter element disposed within the lumen, the first and second catheter elements movable with respect to each other along an axis, wherein the first catheter element includes a first tracking sensor and the second catheter element includes a second tracking sensor. The process comprising tracking a catheter assembly position within the heart based on a first electrical signal from the first tracking sensor and based on a second electrical signal from the second tracking sensor, generating a first position of the first catheter element based on the first electrical signal and generate a second position of the second catheter element based on the second electrical signal, laterally aligning the first position with the second position based on a lateral displacement and a rotational displacement determined from the first position and the second position, and longitudinally adjusting the first location with respect to the second location based on a detection of the first tracking sensor of the second tracking sensor.

In Example 25, the process of Example 24, and further comprising generating an electroanatomical map of the heart and providing the catheter elements with respect to the electroanatomical map of the heart in a visualization.

In Example 26, the process of Example 24, wherein the first catheter assembly position includes a first location in space and a first tangent, and the second catheter assembly position includes a second location in space and a second tangent, and wherein the first location and the first tangent are laterally aligned with the second location and the second tangent based on a lateral departure and a rotational deflection determined from the first tangent and the second tangent.

In Example 27, the process of Example 24, wherein the longitudinally adjusting includes correcting a protrusion via sheath detection of the first catheter element and the second catheter element.

In Example 28, a system for use with an electrophysiological procedure. The system comprising an elongated catheter assembly having a tracking sensor and a controller. The controller is configured to track a plurality of catheter assembly positions within the organ based on a plurality of electrical signals from the tracking sensor, determine a first catheter assembly position based on a first electrical signal of the plurality of electrical signals and determine a second catheter assembly position based on a second electrical signal of the plurality of electrical signals, constrain the second position with the first position, and generate an anatomical map of an organ of the electrophysiological procedure with a visualization of the catheter assembly having the second position constrained with the first position.

In Example 29, the system of Example 28, wherein the controller is configured to place a tag on the anatomical map of the organ at the first catheter assembly position representing an anatomical landmark.

In Example 30, the system of Example 29, wherein the controller is configured to track the catheter assembly independent of the anatomical landmark.

In Example 31, the system of Example 28, wherein the controller is configured to track the plurality of catheter assembly positions of the catheter assembly via the tracking sensor using a tracking methodology as a function of time as it passes through the heart.

In Example 32, the system of Example 31, wherein the controller is configured to constrain the first position to the second position via bending energy data regarding bending energy of the catheter assembly.

In Example 33, the system of Example 28, wherein the controller is configured to constrain the first position to the second position via a path.

In Example 34, the system of Example 33, wherein the controller is configured to highlight the path in the visualization.

In Example 35, the system of Example 28, wherein the catheter assembly including a plurality of coaxially disposed catheter elements, the catheter elements including a first catheter element and a second catheter element, the first catheter element forming an elongated lumen and the second catheter element disposed within the lumen, the first and second catheter elements movable with respect to each other along an axis, wherein the first catheter element includes a first tracking sensor and the second catheter element includes a second tracking sensor, The controller is configured to generate a first position of the first catheter element based on a first electrical signal of the plurality of electrical signals and generate a second position of the second catheter element based on a second electrical signal of the plurality of electrical signals, laterally align the first position with the second position based on a lateral displacement and a rotational displacement determined from the first position and the second position, and longitudinally adjust the first position with respect to the second position based on a detection of the first tracking sensor of the second tracking sensor.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an example clinical setting for treating a patient, and for treating a heart of the patient, the example clinical setting having an example electrophysiology system.

FIG. 2 is a block diagram illustrating an example controller for use with the example electrophysiology system of FIG. 1.

FIG. 3A is a flow diagram illustrating an example configuration of the example controller of FIG. 2.

FIG. 3B is a schematic drawing illustrating an example implementation of the example configuration of FIG. 3A.

FIG. 4 is a flow diagram illustrating another example configuration of the example controller of FIG. 2.

FIG. 5 is a schematic diagram illustrating separated example sections of example catheter elements of an example catheter assembly for use with the example electrophysiology system of FIG. 1.

FIG. 6A is a schematic diagram illustrating a context for an implementation of the process of FIG. 4 with the example catheter elements of FIG. 5.

FIG. 6B is a schematic diagram illustrating a representation of device models from tracking the example of catheter elements of FIG. 6A.

FIG. 6C is a schematic diagram illustrating an implementation of a feature of the process of FIG. 4 with the device models of FIG. 6B.

FIG. 6D is a schematic diagram illustrating an implementation of another feature of the process of FIG. 4 with the device models of FIG. 6C.

While the disclosure is amenable to various modifications and alternative forms, specific embodiments have been shown by way of example in the drawings and are described in detail below. The intention, however, is not to limit the disclosure to the particular embodiments described. Rather, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

Examples of electrophysiological procedures and systems in which electroanatomical mapping systems track catheter assemblies are described in this disclosure with electrophysiological testing and ablation systems for illustration. Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a catheter is inserted into the patient and radio frequency waves are transmitted through the catheter to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques can indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells to increase the permeability of the cell membrane. The electroporation can be reversible or irreversible, depending on the strength and duration of the electric field. If the electroporation is reversible, the temporarily increased permeability of the cell membrane can be used to introduce chemicals, drugs, or deoxyribonucleic acid (DNA) into the cell, prior to the cell healing and recovering. Tissue recovery can occur over minutes, hours, or days after the ablation is completed. If the electroporation is irreversible, the affected cells are killed, such as via some form of cell death, such as perhaps programmed cell death through apoptosis for example, or such as traumatic cell death through necrosis for example.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a relatively safe and effective alternative to the indiscriminate killing of thermal ablation techniques, such as RF ablation and cryoablation. Irreversible electroporation can be used to kill targeted tissue, such as myocardium tissue, by using a selected electric field strength and duration that is effective to kill the targeted tissue but is not effective to permanently damage other cells or tissue, such as non-targeted myocardium tissue, red blood cells, vascular smooth muscle tissue, endothelium tissue, and nerve cells. Irreversible electroporation systems are presented in this disclosure for illustration, but the concepts of catheter and assembly tracking can also apply to other systems.

Cardiac ablation, as well as other electrophysiological procedures, can involve the use of catheter assemblies. A catheter assembly includes a plurality of catheter elements, and catheter elements can include catheters, sheaths, dilators, guidewires, and needles. As electrophysiological procedures move toward less use of fluoroscopy, catheter elements include tracking devices or sensors to facilitate tracking in electroanatomical mapping systems. Typically, catheter elements in a catheter assembly are tracked separately via tracking systems in electroanatomical mapping systems. Because tracking systems are inexact and provide approximations of the positions of the catheter elements within an organ, different catheter elements in a catheter assembly may be rendered in a visualization as displaced or separated from one another even though the catheter assembly incudes one catheter element disposed within another within the patient. It is desirable that the catheter elements be presented in accurate positions with respect to each other and the heart for clinical interpretation. When the tracked elements are physically coaxial, for example, it is desirable that the estimates of the catheter element locations be improved using this information, and that the catheter elements be rendered, such as in a visualization, as coaxial. Similarly, when one of the tracked elements has advanced past another, for example, it is desirable that the catheter elements be rendered as single catheter assembly.

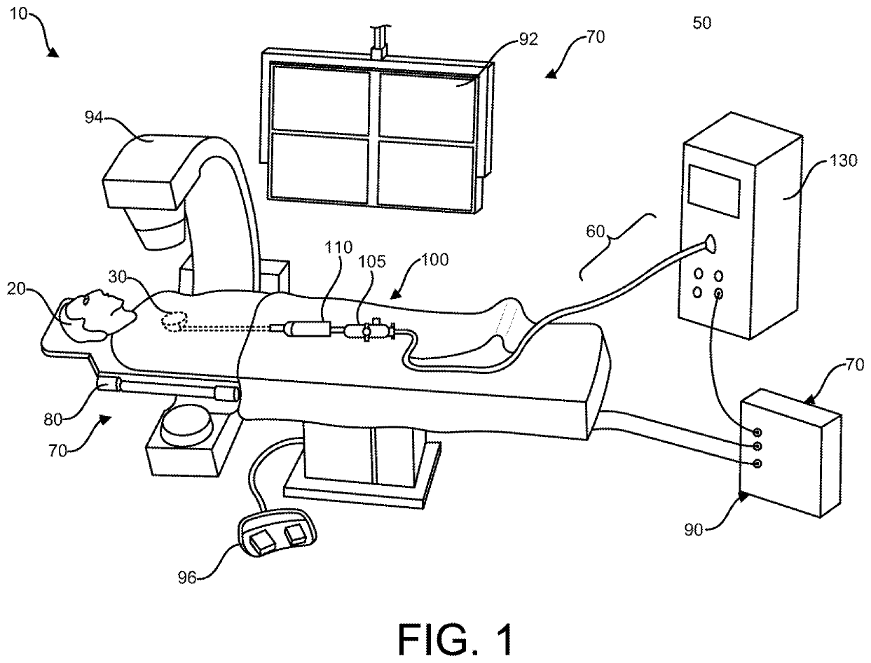

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electroanatomical mapping (EAM) system 70. The example electroporation catheter system 60 includes an elongated catheter assembly 100, which in the example includes an electroporation catheter 105 and an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The electroporation catheter system 60 is configured to deliver electric field energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheter elements. For instance, a catheter element such as a sheath or catheter defines a longitudinal axis that passes through a centroid of a cross section of the catheter element, such as the centroid of a cross section of a catheter shaft or a centroid of a cross section of a lumen of a sheath. Coaxial disposed catheter elements include a catheter element disposed within another catheter element such that the longitudinal axes of each catheter element generally follow the same three-dimensional curve or path up to the most distal point that both are present. The catheter elements can include a first catheter element, such as an elongated introducer sheath 110, and a second catheter element, such as an elongated catheter such as electroporation catheter 105. The first catheter element includes an elongated lumen and the second catheter element is disposed within the lumen. In the example, the catheter 105 is disposed within the introducer sheath 110. The first and second catheter elements are movable with respect to each other along the longitudinal axis. For example, a distal end of the catheter 105 can be manipulated to extend from the distal tip of the introducer sheath 110, or the distal tip of the introducer sheath 110 can be retracted from the distal end of the catheter 105. Additionally, the distal end of the catheter 105 can be retracted from the distal tip of the introducer sheath 110. The first catheter element includes a first tracking sensor, and the second catheter element includes a second tracking sensor. For instance, each of the first and second catheter elements can include one or more tracking sensor. Examples of tracking sensors can include magnetic navigation devices and electrodes.

The introducer sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart. In one example, the catheter assembly 100 including the introducer sheath 110 is adapted for use with a transseptal puncture. The left atrium of the heart is a relatively difficult chamber to access percutaneously, and the transseptal puncture permits a direct route to the left atrium via the intra-atrial septum and systemic venous system.

The example catheter 105 includes an elongated catheter shaft and distal end configured to be deployed proximate to the target tissue, such as within a chamber of the patient's heart. The distal end may include a basket, balloon, spline, configured tip, or other electrode deployment mechanism to effect treatment. The electrode deployment mechanism includes an electrode assembly or array. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. In one example, the plurality of electrodes can be formed of a conductive, solid-surface, biocompatible material and are spaced-apart across insulators. Each of the plurality of electrodes is electrically coupled to a corresponding elongated lead conductor that extends along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to a plug in the proximal region of the catheter 105, such as a plug configured to be mechanically and electrically coupled to the electroporation console 130, for example, either directly or via intermediary electrical conductors such as cabling. In one example, the electroporation console 130 is configured to provide an electrical signal, such as a plurality of concurrent or space-apart-time electrical signals, to the electrically connected catheter 105 along lead conductors to the spaced-apart electrodes. In an example of an electroporation catheter, the spaced-apart electrodes are configured to generate a selected electrical field proximate the target tissue, based on the electrical signals from the electroporation console 130, to effect electroporation.

A selected electrical field can be generated with the electrodes to effect electroporation. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

The electroporation console 130 is configured to control aspects of the electroporation catheter system 60. In embodiments, the electroporation console 130 is configured to provide one or more of the following: modeling the electric fields that can be generated by the electroporation catheter 105, which often includes consideration of the physical characteristics of the electroporation catheter 105 including the electrodes and spatial relationships of the electrodes on the electroporation catheter 105 and, whether the electroporation catheter 105 is in bipolar or monopolar mode; generating the graphical representations of the electric fields, which often includes consideration of the position of the electroporation catheter 105 in the patient 20 and characteristics of the surrounding tissue; and overlaying, on the display 92, the generated graphical representations on an anatomical map. In some examples, the electroporation control console 130 is configured to generate the anatomical map. In some examples, the EAM system 70 is configured to generate the anatomical map for display on the display 92.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric fields induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

An example EAM system 70 tracks locations of catheter elements of a catheter assembly 100 using processes known in the art. In one example of an EAM system 70 provides equations that include the position and perhaps velocity of each of the electrodes. Tracked devices, such as catheter elements of the catheter assembly 100, are characterized in the EAM system 70 as device models that include electrode locations and tangents among other parameters. In one example presented for illustration, tracking can include the application of external electrodes that generate a plurality of non-parallel electric fields throughout the body for impedance tracking. A magnetically tracked device is used to associate those electric field values, or impedance values, with each position in space as determined magnetically. A resulting data structure is presented as a field map. In the case of only an impedance device being tracked, the field map is inverted to estimate the location of each electrode. The estimated locations are smoothed, and the processes include a variety of constraints on the shape of the flexible device comprised of all its electrodes taken together. The body is nearly transparent to magnetic fields, but the electrical properties of body tissues affect the shape of the electric fields dramatically, impedance tracking is less accurate than magnetic tracking.

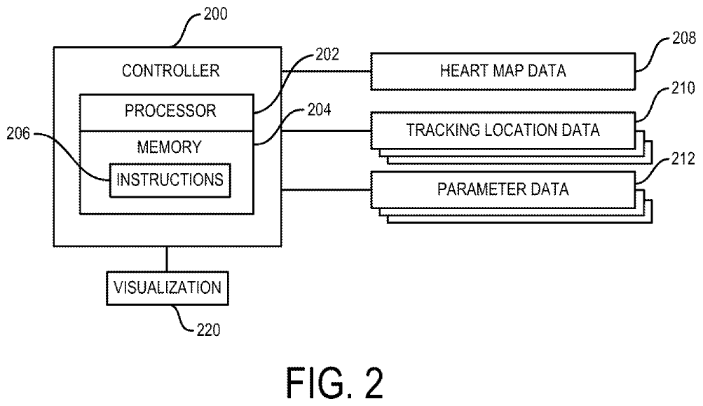

FIG. 2 illustrates an example controller 200 that can be used with the example electrophysiology system 50, such as a controller of the example electroporation catheter system 60, which may include a controller of the example EAM system 70 such as the mapping and navigation controller 90. The controller 200 can be implemented to provide a device model and visualization of a catheter assembly as a single unit within a patient even when the catheter elements of the catheter assembly are independently tracked and independently tracked with different methodologies during an electrophysiological procedure. The controller 200 can include a processor 202 and a memory 204. The memory 204 stores processor executable instructions 206. In one example, the processor executable instructions can be in the form of a program, such as a computer program or application. The processor 202 can execute the instructions 206 that can be included in configuring the controller 200. In one example, the controller 200 can be implemented to include a computing device such as a laptop computer, a workstation, a desktop computer, a tablet, or a smartphone. In such examples, the controller 200 can include additional components such as a display, a touchscreen, speakers or other output devices, a keyboard or other input devices, or communication circuitry such as computer network adapters. The controller 200 may be implemented in a variety of architectures and components, such as the processor 202 and memory 204, may be distributed in various locations.

In one example, the processor 202 may include a plurality of main processing cores to run an operating system and perform general-purpose tasks on an integrated circuit. The processor 202 may also include built-in logic or a programmable functional unit, also on the same integrated circuit with a heterogeneous instruction-set architecture. In additional to multiple general-purpose, main processing cores and the application processing unit, controller 200 can include other devices or circuits such as graphics processing units or neural network processing units, which may include heterogeneous or homogenous instruction set architectures with the main processing cores. For example, the controller 200 may be used to perform other tasks such as in the case of a computing device including the resonance sound amplification device.

Memory 204 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 202. Any such computer storage media may be part of the controller 200 and implemented as memory 204. Memory 204 is a non-transitory, processor readable memory device. Accordingly, a propagating signal by itself does not qualify as storage media or memory 204.

The controller 200 may be configured to receive inputs or information from the electrophysiology system 50, such as inputs from the electroporation catheter system 60 and EAM system 70 including the electroporation console 130 and the mapping and navigation controller 90, for storage in memory 204 and use by the instructions 206. For example, the controller 200 can receive an input representative of the anatomical map of the heart, or heart map data 208, which heart map data 208 can include the data regarding representations of the geometric anatomical map of the heart and the electro-anatomical map of the heart, such as from the EAM system 70. Additionally, heart map data 208 can include annotations, markings, or user-added tags of the heart that may include markings of anatomical locations of interest or other data to generate visualizations a clinician may find of interest during a procedure. The controller 200 can also receive tracking location data 210 for each catheter element of the catheter assembly. The tracking location data can be generated by a magnetic tracking techniques, impedance tracking methodologies, or other tracking procedures, and can be generated via the EAM system 70 or other features of electrophysiology system 50. In one example, tracking location data 210 includes information used to determine the position of sensors on the catheter elements with respect to the heart. Also, tracking location data 210 can include information regarding how many electrodes in the catheter are exposed from underneath the sheath, such has sheath detection data if the electrophysiology system 50 includes sheath detection mechanisms. In some examples, the processor 200 can receive catheter assembly parameter data 212, including information regarding the parameters of the catheter elements such as number and spacing of electrodes on catheter element, the type of tracking methodology used, and various other parameters such as mechanical properties and state, such as, in one embodiment, properties related to bending or bending energy, of the catheter elements that may be used to determine constraints. In one catheter assembly parameter data 212 can include a separate input for each catheter element. In one example, heart map data 208 and tracking location data 210 are stored in memory 204 for use by the processor 202 executing the instructions 206.

The controller 200 is configured to generate a visualization 220 that can include determined location of the catheter assembly as a single unit based on the received tracking data of the catheter elements with reference to the anatomical map of the heart. In one example, the controller 200 is configured to generate a visualization 220 based on constraints applied to the tracking data 210 and catheter parameter data 212 with heart map data 208.

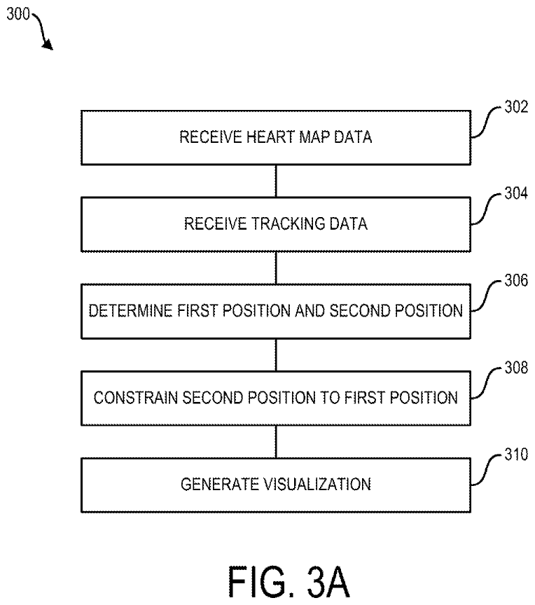

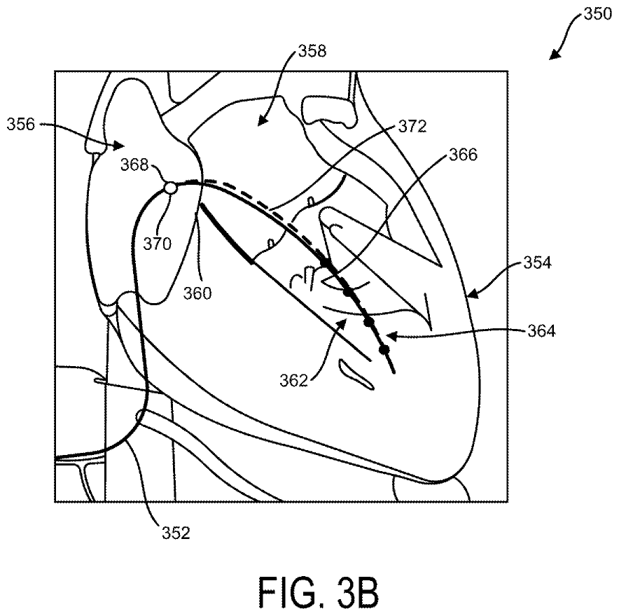

FIG. 3A illustrates a process 300 of configuring a controller, such as controller 200, while tracking a catheter assembly having a catheter element with a tracking sensor. Process 300 includes configuring the controller to receive electroanatomical map data of an organ, such as receive heart map data 208, at 302. The controller is configured to track a plurality of catheter assembly positions within the organ based on a plurality of electrical signals based on the tracking sensor at 304. For example, the controller can track the location of the catheter assembly via the tracking sensor using a tracking methodology as a function of time as it passes through the heart. In another example, a plurality of tracking signals can be generated concurrently, or generally concurrently, such as a plurality of tracking sensors of the catheter assembly can be tracked in the same sample. The controller is configured to determine a first catheter assembly position based on a first electrical signal of the plurality of electrical signals and determine a second catheter assembly position based on a second electrical signal of the plurality of electrical signals at 306. The first and second catheter assembly positions can each include a set of data determined by the mapping and navigation controller 90 regarding the catheter element in space, such as the catheter pose or the three-dimensional curve of the distal portion of the catheter having the tracking sensor as included in tracking location data 210. In one example, the first catheter assembly position includes a first location in space and a first tangent of catheter assembly as determined by the EAM system 70, and the second catheter assembly position includes a second location in space and a second tangent of catheter assembly as determined by the EAM system 70. The second position is constrained with the first position at 308. In one example, catheter parameter data 212 is applied to the tracking location data 210 to constrain the positions. The controller is configured to generate an electroanatomical map of an organ of the electrophysiological procedure with a visualization of the catheter assembly having the second position constrained with the first position at 310.

In one example, process 300 can be implemented as set of processor-executable instructions, such as instructions 206, stored in a non-transitory memory, such as memory 204 to be executed by a processor 202 to configure controller 200. The instructions to implement process 300 can be configured to receive information, such as to retrieve from memory 204, heart map data 208 and tracking location data 210 and catheter parameter data 212 for a catheter element including a plurality of catheter elements. Further, the instructions to implement process 300 can be configured to annotate, adjust, or write to heart map data 208 and to generate a visualization, such as visualization 220 on a display of graphical representations.

The process 300 provides for catheter assembly or catheter element visualization and tracking. In one example, the process 300 can be implemented to combine anatomical landmarks and catheter mechanics for catheter visualization and tracking. As catheter assemblies are tracked in real time, the current position of the catheter assembly in the heart is presented as a visualization.

FIG. 3B provides a simplified schematic view 350 of the process 300 implemented to combine anatomical landmarks and catheter mechanics for catheter visualization and tracking. A distal region of catheter 352 is illustrated as having advanced through a patient's vasculature and into the heart 354. The heart 354 includes a right atrium 356, left atrium 358, and intra-atrial septum 360. The distal region of catheter 352 includes a tracking sensor 362, which in the example includes a plurality of electrodes 364 disposed along a shaft 366. In the process 300, anatomical landmarks are preserved with tags as a catheter advances past the landmark, such as at 304. In one example, the catheter 352 may cross the intra-atrial septum 360 from the right atrium 356 to the left atrium 358 at a crossing point 368 of the intra-atrial septum 360. A tag 370 can be placed manually by the operator or automatically at the anatomical landmark of interest with process 300 at 304. The electrodes 364 are tracked on a distal region of the catheter at 306. The shape of the catheter shaft 366 from the tag 370 to the tracked electrodes 364 generally follows a path to reduce the mechanical bending energy of the shaft. Process 300 determines the catheter position, such as based on the location of tag 370 and the electrodes 364, and imposes constraints related to the shape of the catheter in a device model, at 308. A dashed line represents the device model 372. Bending energy of the catheter, a parameter determinable via catheter parameter data 212, can be applied along with other constraints to determine a catheter trajectory in the device model 372.

A visualization produced at 310 can highlight the anatomical region of the intra-atrial septum crossing with a marker or a selected color, a selected color indicating the location of the electrodes with respect to a heart map, and another color as a line, arc, or spline to passing through the marker and the electrodes following a shape that is constrained to the catheter location based on the catheter parameter data 212. In one example, the catheter element is tracked independently of the anatomical landmark, and the curve or path with reduced bending energy is also determined independently. In another example, the catheter element is tracked with additional constraints based on both the anatomical landmark and the bending energy from catheter parameter data 212.

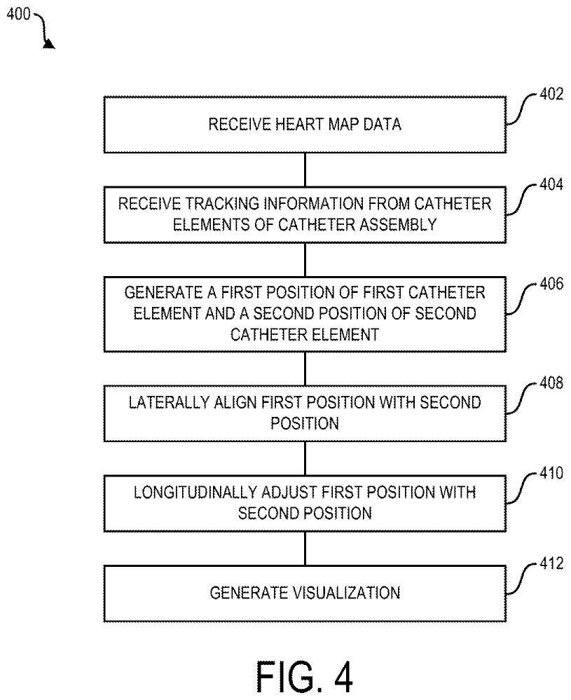

FIG. 4 illustrates a process 400 of configuring a controller, such as controller 200, while tracking a plurality of catheter elements each having tracking sensors of the catheter assembly. For instance, process 400 can be an example of process 300 of FIG. 3. In the example process 400, the catheter assembly includes a plurality of coaxially disposed catheter elements, and the catheter elements including a first catheter element and a second catheter element. The first catheter element forms an elongated lumen, and the second catheter element is disposed within the lumen. The first and second catheter elements are movable with respect to each other along an axis. An example of first catheter element can be a sheath, and an example of second catheter element can be a catheter disposed within a lumen of the sheath. The first catheter element includes a first tracking sensor, and the second catheter element includes a second tracking sensor. Process 400 can receive parameters regarding the first catheter element and second catheter elements via catheter parameter data 212.

Process 400 includes configuring the controller to receive electroanatomical map data of an organ, such as receive heart map data 208, at 402. The controller is configured to track a catheter assembly position within the heart based on a first electrical signal from the first tracking sensor and based on a second electrical signal from the second tracking sensor at 404. In the example, the positions of the catheter elements are tracked generally concurrently. The controller is configured to generate a first position of the first catheter element based on the first electrical signal and to generate a second position of the second catheter element based on the second electrical signal at 406. For instance, the controller is configured to generate the position of each electrode on the first catheter element based on its set of magnetic or electrical signals, and similarly for the second catheter element. The first and second catheter element positions can each include a set of data determined by the mapping and navigation controller 90 regarding the catheter element in space, such as the catheter pose or the three-dimensional curve of the distal portion of the catheter having the tracking sensor as included in tracking location data 210. In one example, the first catheter element position includes a first location in space and a first tangent of the catheter element as determined by the EAM system 70, and the second catheter element position includes a second location in space and a second tangent of the catheter element as determined by the EAM system 70. In an example as used in this disclosure, two objects are considered coaxial at a point if the objects touch at the point and have a same tangent at the point. The first position is laterally aligned with the second position based on a lateral displacement of the first catheter element relative to the second catheter element at 408. For example, the first location and the first tangent are laterally aligned with the second location. and the second tangent based on a lateral displacement and a rotational displacement determined from the first location and first tangent and the second location and second tangent, respectively. For example, the tangents at the point can be aligned by a rigid body transformation such as rotation or by using points along one device to modify locally the tangent of the other device. The first position is longitudinally adjusted with respect to the second position based on a detection of the second tracking sensor using the first tracking sensor at 410. For example, the first position is longitudinally aligned with respect to the second position based on a detection of the first tracking sensor of the second tracking sensor. The controller is configured to generate an electroanatomical map of an organ of the electrophysiological procedure with a visualization of the catheter assembly having a device model of the first catheter element constrained with a device model of the second catheter element at 412.

Lateral alignment at 408 and longitudinal adjustment at 410 can be performed based on various determinations from the tracking location data 210 and catheter parameter data 212. Lateral alignment at 408 can be applied to correct departure or displacement in the device models of the catheter elements. In one example, departure or displacement is addressed via translation, or moving along a vector from one point to another. Further, lateral alignment at 408 can be applied to correct deflection, or rotation of the device models of the catheter elements. in one example, rotation is applied by moving by an angle. Longitudinal adjustment at 410 can be applied to correct protrusion, or longitudinal displacement of one catheter element with respect to the other catheter element. A determination of the number of electrodes exposed or uncovered by the sheath can be used to provide the longitudinal adjustment. In one example of a tracked catheter and sheath, sheath detection—or the technology used to determine whether and how many electrodes of the catheter are exposed from under the sheath—is used to place constraints on the relative locations of the device models of the catheter and sheath, such as for longitudinal adjustment at 410. Rotational alignment at can be applied to align the local tangents by rotating one or both objects or by using one object to modify the local shape of the other object. In another example of 408, the positions and poses of the catheter elements can be determined by tracking the catheter elements of the catheter assembly, applying a likely curve of the catheter assembly using reduced bending energy as determined from catheter parameter data 212, and finding the positions and poses again but constrained to the likely curve. This example process may be repeated.

One or more catheter of the devices models of the catheter elements can be aligned and adjusted. The determinations of lateral alignment at 408 and longitudinal adjustment at 410 can assign weights to each of the device models of the catheter elements based on the confidence that the tracked catheter element is in the site in space with respect to the heart as represented in the tracking. In general, the confidence in the tracking of the two elements can be used to weight the operations applied to each element to affect the desired constraints. For example, magnetic tracking is relatively accurate and closely represents the location of the catheter element site in space with respect to the heart. In the case of a catheter configured for magnetic tracking and the sheath configured for impedance tracking, an approach is to give device model of the catheter a high confidence value (e.g., 1.0) and the device model of the sheath is laterally aligned with the tracked position of the device model of the catheter given the relative accuracy of magnetic tracking. In the case of a catheter configured for impedance tracking and the sheath configured for impedance tracking, the device model of the catheter can be given a confidence value of less than 1.0 and the device model of the sheath is given a confidence value of less than 1.0 as based on information from the catheter parameter data regarding the likelihood of accurate tracking.

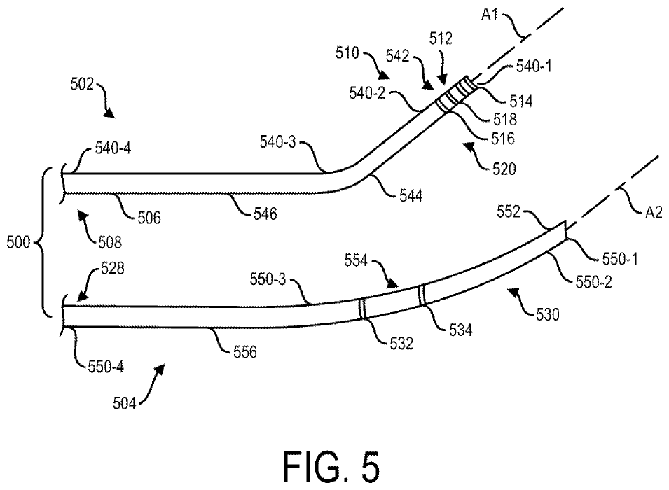

FIG. 5 illustrates an example catheter assembly 500 having a plurality of separate distal sections of catheter elements including a catheter 502 and a sheath 504, which can be an example of catheter 105 and sheath 110 of FIG. 1. Distal section of catheter 502 includes a shaft 506 disposed on a longitudinal axis A1 and having a proximal region 508 and a distal region 510. The distal region 510 includes a plurality of electrodes 512 for ablating tissue. The plurality of electrodes 512 in the example includes a tip electrode 514 and ring electrodes 516, 518 carried on a distal portion 520 of the shaft 506. The catheter shaft 506 can be flexible along its entire length of the distal section, but in the example shown, the distal portion 520 carrying the electrodes 512 is rigid and not bendable. The catheter shaft 506 can include a navigation device (not shown) in or near the distal portion 520 within the shaft 506 for magnetic tracking. The distal section of the sheath 504 can define a lumen along longitudinal axis A2 such that the catheter 502 can be carried inside the lumen. The distal section of the sheath 504 includes a proximal region 528 and a distal region 530. The distal region 530 includes a plurality of ring electrodes 532, 534 for impedance tracking as well as sheath detection of catheter electrodes 512. In one example, the electrophysiology system 50 applies sheath detection to determine whether the catheter distal portion 520 is disposed within the sheath 504 and/or the number of catheter electrodes 512 that are exposed from underneath the sheath 504. An example of sheath detection is described in U.S. patent application Ser. No. 16/686,591, titled SHEATH DETECTION USING LOCAL IMPEDANCE INFORMATION, filed Nov. 18, 2019, to Salehi et al., and assigned to the present assignee, the contents of which are incorporated by reference into this disclosure to the extent it is consistent with this disclosure. In this example, the distal section of the sheath 504 is flexible along its entire length including along the sheath distal region 530 with the exception, possibly, of a relatively small distal-most portion.

The EAM system 70 generates device models of the distal sections of the catheter elements 502, 504 for processing. In one example, the device models include data from the distal tip of the catheter element to a proximal bound of an articulable segment of the catheter element, or distal section. For example, a device model for each catheter element 502, 504 includes a point and three device segments. The point is a tip of the catheter element. The distal-most point of the catheter element is the tip, such as catheter tip 540-1 and sheath tip 550-1. The device segments of the device model of the catheter element include a head segment, a neck segment, and a body segment. The head segment 542, 552 includes the distal-most rigid tip segments of the catheter element. On the catheter 502, the head segment 542 is between 540-1 and 540-2. On the sheath, the head segment 552 is between 550-1 and 550-2. The neck segment 544, 554 includes the flexible articulable segment proximal to the head segment 542, 552, respectively. On the catheter 502, the neck segment 544 is between 540-2 and 540-3. On the sheath 504, the neck segment 554 is between 550-2 and 550-3. The body segment 546, 556 includes the flexible segment most proximal on the catheter element. On the catheter 502, the body segment 546 is between 540-3 and 540-4. On the sheath 504, the body segment 556 is between 550-3 and 550-4.

Based on values of the electrode locations and electrode tangents, the EAM system can constrain each device segment of the device model into a geometric shape. In one example, a catheter element used in an electrophysiology procedure is classified as either a rigid device or a flexible device. A rigid device has all the electrodes on the head segment, such as example catheter 502, and a flexible device has all electrodes on a neck segment, such as example sheath 504. The geometric shapes used to represent the device segments can include a point, line, circle, any other defined geometric shape, or a spline. For a rigid device, such as catheter 502, a device model fits as follows: (a) the head segment 542 is fit as line to the electrodes 512, (b) the tip 540-1 is fit as a point at the end of the head segment 542, and (c) the neck segment 544 is fit as spline (if, the neck of the catheter 502 is extended from the sheath 504). For a flexible device, such as sheath 504, a device model fits as follows: (a) the neck segment 554 is fit as a spline (in other examples, the segment can be fit as a circle or line), (b) the head segment 552 is fit as a line using the tangent of the distal most electrode 532, and (c) the tip 550-1 is fit as a point at the end of the head segment 552. For a flexible device, a spline fit to the electrodes can take the shortest path that connects the electrodes subject to smooth constraints such as continuity of the first and second spatial derivatives. This path, however, may not satisfy the known distance between electrodes for example. In order to meet this constraint, a simple geometric object such as a circle can be used to constrain the spline over some or all of the flexible device segment. In one example, a geometric object such as a circle may be used to add additional points that constrain a spline to be fit using only points. In another example, a geometric object such as a circle may be used to compute tangents that constrain a spline to be fit using both points and tangents. In another example, a geometric object such as a circle may be used to compute tangents and distances between electrodes that constrain a spline to be fit using points, tangents and distanced between electrodes.

In the following examples, the catheter 502 is tracked via magnetic tracking and the sheath 504 is tracked via impedance tracking, and the tracking of the catheter is given a confidence value of 1.0, so the during the application of process 400, the device model of the sheath 504 is adjusted and aligned to fit the model of the catheter 502. In another embodiment the tracking of the sheath is given a confidence value of 1.0, so the device model of the catheter is adjusted to align and fit the model of the sheath. In another embodiment both devices are given confidence values between 0.0 and 1.0, due to noise levels or other metrics of tracking accuracy, so the two device models are shifted toward each other, e.g., toward some noise-weighted average. Also, the catheter assembly 500 implements sheath detection.

Sheath detection data can be included with catheter location data 210 as including a state or a number of exposed electrodes on the catheter 502. For instance, sheath detection data can report a covered state, a partially covered state, and an uncovered state. A covered state is when the sheath 504 entirely covers a catheter 502, such as when the sheath 504 covers all the electrodes 512 on the catheter 502. A partially covered state is when some, but not all, of the electrodes 512 on a catheter 502 are exposed from underneath the sheath 504. An uncovered state is when all the electrodes 512 of the catheter 502 are exposed from underneath the sheath 504. In other embodiments, the coverage state may be determined by purely geometric means that quantify the geometric relations between the two device models. In other cases, the coverage state may be determined initially by input from the users, and state transitions may be achieved by a state machine that uses geometric information to control transitions between neighboring states.

In a covered state, constraints are applied to the catheter elements if the catheter tip 540-1 is advanced beyond (distal) the most proximal sheath electrode 532. A point on the sheath body 556 closest to the tip of the catheter head 542 is determined. The device model of the sheath body 556 is translated to be coaxial with the nearest point of the device model of the catheter head 542. In terms of coaxial in space, the two device models are constrained to intersect at that point and have parallel tangents at that point. Also, the sheath tip 550-1 is adjusted to be consistent with the number of exposed electrodes 512, which is zero in the covered case. To translate the device model of the sheath 504 to the device model of the catheter 502 in the covered state, which includes a transformation of a vector from one point to another point, the device model of the sheath bod 556 is moved laterally to the device model of the catheter head 542 and is moved longitudinally to satisfy the sheath detection of no exposed electrodes. The device model of the sheath 504 is also rotated or otherwise modified about the tip of the device model of the catheter to match local tangents of the catheter 502.

In a partially covered state, the device model of the sheath head 552 is translated to be coaxial with the device model of the catheter head 542, and the device model of the sheath tip 550-1 is translated to expose the number of electrodes indicated by the sheath detection data. To translate the device model of the sheath 504 to the device model of the catheter 504 in the partially covered state, which includes a transformation of a vector from one point to another point, the shortest vector between the device models of the sheath tip 550-1 and catheter head 542 defines a translation. The device model of the sheath 504 is moved longitudinally to satisfy the sheath detection data of the proper number of exposed electrodes. The device model of the sheath tip 550-1 is also rotated or otherwise modified about the device model of the catheter tip 540-1 to match local tangents of the catheter tip 540-1.

In an uncovered state, constraints are applied to the device models of the catheter elements 502, 504 if the catheter tip 540-1 protrusion is less than a bound of the catheter neck 544. The device model of the sheath head 552 is translated to be consistent with the articulation of the device model of the catheter neck 544. Also, the device model of the sheath tip 550-1 is adjusted to be consistent with the number of exposed electrodes 512, which is all the electrodes in the uncovered case. To translate the device models of the sheath 504 to the catheter 502 in the covered state, which includes a transformation of a vector from one point to another point, the device model of the sheath tip 550-1 moved laterally to the device model of the catheter head 542, and the device model of the sheath tip 550-1 is moved longitudinally to satisfy the sheath detection of all exposed electrodes. The device model of the sheath tip 550-1 is also rotated or otherwise modified about the device model of the catheter tip 540-1 to match local tangents.

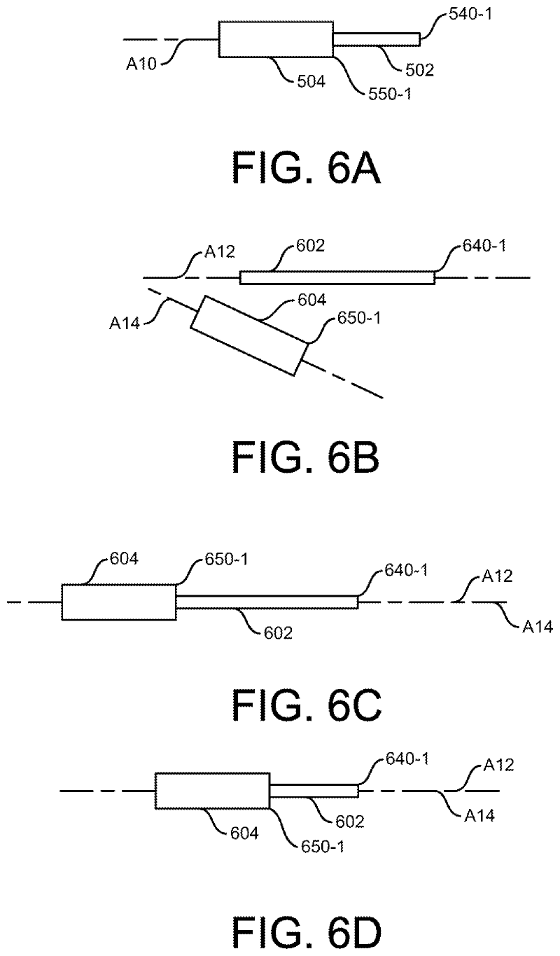

FIG. 6A-6D illustrate an example implementation of the process 400 as applied to catheter assembly 500. In the illustrated example implementation, the catheter 502 is tracked via magnetic tracking and the sheath 504 is tracked via impedance tracking. In this case, the tracking of the catheter 502 is given a confidence value of 1.0, so the during the application of process 400, the device model of the sheath 504 is adjusted and aligned to fit the determined position of the device model of the catheter 502. Also, the electrophysiology system 50 includes sheath detection technology and the process 400 implements use of sheath detection data as part of catheter location data 210.

FIG. 6A illustrates the actual location of the catheter assembly 500 in space and deployed within a heart chamber. The catheter assembly 500 includes a including a plurality of coaxially disposed catheter elements such as the catheter 502 and sheath 504. The sheath 504 forms an elongated lumen and the catheter 502 is disposed within the lumen. The catheter 502 and sheath 504 are movable with respect to each other along an axis A10, and, as illustrated, the catheter 502 juts from sheath 504 such that the catheter tip 540-1 is extended past the sheath tip 550-1.

FIG. 6B illustrates a visual representation of the device model catheter assembly 600 constructed because of the independent tracking of the catheter elements 502, 504 prior to application of the process 400. The device model catheter assembly 600 includes a device model of the catheter 602 and a device model of the sheath 604 corresponding with the catheter 502 and sheath 504, respectively. FIG. 6B is an exaggerated representation of the divergence of the device models of the catheter elements 602, 604 for illustration. The device model of the catheter 602 includes a device model catheter tip 640-1, and the device model of the sheath 604 includes a device model sheath tip 650-1. As illustrated, the device model of the catheter 602 is laterally displaced from the device model of the sheath 604 in departure prior to the application of process 400. Also as illustrated, the device model of the catheter 602 is disposed along axis A12 and the device model of catheter 604 is disposed along axis A14, in which axes A12 and A14 are not coincident but are deflected via rotational displacement prior to the application of process 400. Despite that the catheter assembly 500 is coaxial within the heart, the device models of the catheter 602 and sheath 604 appear to float and appear to be in different positions in space according to the independent tracking. The process 400 will laterally align the device model of the sheath 604 with the device model of the catheter 602 at the position of the device model of the catheter 602. For instance, the axes A12, A14 of the device models 602, 604 in the illustration will be made parallel and then overlapping. Process 400 will longitudinally adjust the align the device model of the sheath 604 with the device model of the catheter 602 at the position of the device model of the catheter 602.

FIG. 6C illustrates a visual representation of the device model catheter 602 and device model sheath laterally aligned in process 400, such as at 408. In the example, the position of the device model of the sheath 604 is translated to the position of the catheter 602 to correct departure. Additionally, the device model of the sheath 604 is rotated to the position of the catheter 602 to correct deflection. In the example, axes A12 and A14 are now coincident to intersect and include parallel tangents. Also as illustrated, the device model of the catheter 602 protrudes, or is still longitudinally or axially displaced, from the device model of the sheath 604.

FIG. 6D illustrates a visual representation of the device model catheter 602 and device model sheath longitudinally aligned in process 400, such as at 410. In the example, the position of the device model of the sheath 604 is translated on axis A14 to the position of the catheter 602 to correct protrusion, which may be based on sheath detection data. As indicated, axes A12 and 14 are coincident. Departure, deflection, and protrusion are corrected as illustrated in FIG. 6D, and the device model of the catheter assembly 600 can be provided for presentation in a visualization at 412.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for use with an electrophysiological procedure, the system comprising:
an elongated catheter assembly including a plurality of coaxially disposed catheter elements, the catheter elements including a first catheter element and a second catheter element, the first catheter element forming an elongated lumen and the second catheter element disposed within the lumen, the first and second catheter elements movable with respect to each other along an axis, wherein the first catheter element includes a first tracking sensor and the second catheter element includes a second tracking sensor; and
a controller configured to:
determine an anatomical map of a heart of the electrophysiological procedure and track a catheter assembly position within the organ based on a first electrical signal from the first tracking sensor and based on a second electrical signal from the second tracking sensor;
generate a first position of the first catheter element based on the first electrical signal and generate a second position of the second catheter element based on the second electrical signal;
laterally align the first position with the second position based on a lateral displacement and a rotational displacement determined from the first position and the second position; and
longitudinally adjust the first location with respect to the second location based on a detection of the first tracking sensor of the second tracking sensor.

2. The system of claim 1, and further including the controller configured to generate a visualization with the catheter elements with respect to the anatomical map of the heart after laterally aligning and longitudinally adjusting.

3. The system of claim 1, wherein the first catheter assembly position includes a first location in space and a first tangent, and the second catheter assembly position includes a second location in space and a second tangent.

4. The system of claim 3, wherein the first location and the first tangent are laterally aligned with the second location and the second tangent based on a lateral departure and a rotational deflection determined from the first tangent and the second tangent.

5. The system of claim 1, wherein the controller is configured to longitudinally adjust the first location with respect to the second location based on a sheath detection.

6. The system of claim 1, wherein the controller is configured to apply a confidence value to the second position.

7. The system of claim 6, wherein the confidence value is 1.0 if the second catheter element is magnetically tracked in an electrophysiology system.

8. The system of claim 1, wherein the first catheter element is a sheath and the second catheter element is a catheter disposed within the sheath.

9. A process for use with an elongate catheter assembly during an electrophysiological procedure on the heart, the catheter assembly including a plurality of coaxially disposed catheter elements, the catheter elements including a first catheter element and a second catheter element, the first catheter element forming an elongated lumen and the second catheter element disposed within the lumen, the first and second catheter elements movable with respect to each other along an axis, wherein the first catheter element includes a first tracking sensor and the second catheter element includes a second tracking sensor, the process comprising:
tracking a catheter assembly position within the heart based on a first electrical signal from the first tracking sensor and based on a second electrical signal from the second tracking sensor;
generating a first position of the first catheter element based on the first electrical signal and generate a second position of the second catheter element based on the second electrical signal;
laterally aligning the first position with the second position based on a lateral displacement and a rotational displacement determined from the first position and the second position; and
longitudinally adjusting the first location with respect to the second location based on a detection of the first tracking sensor of the second tracking sensor.

10. The process of claim 9, and further comprising generating an electroanatomical map of the heart and providing the catheter elements with respect to the electroanatomical map of the heart in a visualization.

11. The process of claim 9, wherein the first catheter assembly position includes a first location in space and a first tangent, and the second catheter assembly position includes a second location in space and a second tangent, and wherein the first location and the first tangent are laterally aligned with the second location and the second tangent based on a lateral departure and a rotational deflection determined from the first tangent and the second tangent.

12. The process of claim 9, wherein the longitudinally adjusting includes correcting a protrusion via sheath detection of the first catheter element and the second catheter element.

13. A system for use with an electrophysiological procedure, the system comprising:
an elongated catheter assembly having a tracking sensor; and
a controller configured to:
track a plurality of catheter assembly positions within the organ based on a plurality of electrical signals from the tracking sensor;
determine a first catheter assembly position based on a first electrical signal of the plurality of electrical signals and determine a second catheter assembly position based on a second electrical signal of the plurality of electrical signals;
constrain the second position with the first position; and
generate an anatomical map of an organ of the electrophysiological procedure with a visualization of the catheter assembly having the second position constrained with the first position.

14. The system of claim 13, wherein the controller is configured to place a tag on the anatomical map of the organ at the first catheter assembly position representing an anatomical landmark.

15. The system of claim 14, wherein the controller is configured to track the catheter assembly independent of the anatomical landmark.

16. The system of claim 13, wherein the controller is configured to track the plurality of catheter assembly positions of the catheter assembly via the tracking sensor using a tracking methodology as a function of time as it passes through the heart.

17. The system of claim 16, wherein the controller is configured to constrain the first position to the second position via bending energy data regarding bending energy of the catheter assembly.

18. The system of claim 13, wherein the controller is configured to constrain the first position to the second position via a path.

19. The system of claim 18, wherein the controller is configured to highlight the path in the visualization.

20. The system of claim 13, wherein
the catheter assembly including a plurality of coaxially disposed catheter elements, the catheter elements including a first catheter element and a second catheter element, the first catheter element forming an elongated lumen and the second catheter element disposed within the lumen, the first and second catheter elements movable with respect to each other along an axis, wherein the first catheter element includes a first tracking sensor and the second catheter element includes a second tracking sensor; and
wherein the controller is configured to:
generate a first position of the first catheter element based on a first electrical signal of the plurality of electrical signals and generate a second position of the second catheter element based on a second electrical signal of the plurality of electrical signals;
laterally align the first position with the second position based on a lateral displacement and a rotational displacement determined from the first position and the second position; and
longitudinally adjust the first position with respect to the second position based on a detection of the first tracking sensor of the second tracking sensor.
